(19) United States
(12) Patent Application Publication
(10) Pub. No.: US 2002/0151848 A1
(43) Pub. Date: Oct. 17, 2002

(54) Protective cover for an elongated instrument

(76) Inventors: Dagoberto T. Capote JR., Memphis, TN (US); Cynthia Lee Corbell, Jacksonville, FL (US)

Correspondence Address: THOMPSON & KNIGHT, L.L.P., PATENT PROSECUTION GROUP, 1700 PACIFIC AVENUE, SUITE 3300, DALLAS, TX, 75201, US

(21) Appl. No.: 09/832,669

(22) Filed: Apr. 11, 2001

(51) Int. Cl. A61M 5/00

(52) U.S. Cl. 604/171; 604/263

(57) ABSTRACT

A protective cover (20, 60, 190) for an elongated instrument comprising a hollow elongated member (22, 78, 92, 100, 124, 168, 178, 186, 200) and at least one loop member (28, 30, 62, 94, 122, 130, 134, 150, 160, 174, 182, 192, 194). The elongated member has a first and second end at opposite ends thereof. At least a portion of the elongated member is a pliable material. A loop member extends along a generally circumferential direction or around a perimeter of the elongated member at the first end. The loop member is affixed to and supports the elongated member along at least a portion of a circumference or perimeter of the elongated member such that an opening is formed at the first end of the elongated member, and such that when in a relaxed configuration, the opening remains open without a force being applied to the loop member. The proportions of the height and longitudinal dimensions of the loop member cross-section are such that the loop member has enough structural strength the maintain the opening at the first end, even in a relaxed state.

### FIELD OF THE INVENTION

The present invention relates to a protective cover for an elongated instrument.

### DESCRIPTION OF RELATED ART

A surgery room, as found in a typical hospital operating room, is an environmentally controlled area where it is desired to minimize the risk of infection from naturally occurring organisms and other contaminants. However, such surgery rooms are often exposed to bodily fluids, which may contain communicable diseases. The cleanliness of a surgery room environment is typically controlled by limiting the introduction of infection-creating organisms and other contaminants with strict cleanliness control of personnel and equipment present in the surgery room.

One way to minimize the risk of infection to patients during surgery in an operating room is by using protective covers on the personnel and equipment. The protective covers are generally placed over the patient, operating room personnel, and operating room equipment to create a sterile barrier to hinder organisms and contaminants that could cause infections from migrating to exposed tissue, organs, or open wounds. Also, the protective covers prevent most bodily fluids, such as blood or lymphatic fluids, that are encountered during most surgical procedures from settling on operating room furniture and equipment. Such bodily fluids may become airborne when, for example, a vein or artery is severed. In some cases, these bodily fluids may contain contaminants or communicable diseases, such as hepatitis or staphylococcus.

The advancement of medical procedures has correspondingly increased the demand for more advanced medical equipment within the operating room. As surgical procedures are becoming more complex and addressing ever smaller parts of the human anatomy, surgical microscopes, cameras, and lasers are now often used in the operating room during surgery. Also, advancements in diagnostic methods and equipment has increased the use of expensive and complex cameras with telescopic lens, tube extensions with prisms therein, and/or small fiber-optic lens extensions for probing within a person. Such advancements in surgical instruments and technology typically means more expensive instruments. Also, such equipment is typically made to be handheld and easy for a surgeon to handle and hold, which leads to the use of power and communications cords extending between the handheld device and a larger unit (e.g., a measurement instrument or computer system) that drives the handheld device. Such power and communications cords make the handheld instruments become an elongated instrument. Also, such cords are often extended along the floor so that they do not become obstacles to the personnel in the operating room, which inevitably leads to them being stepped on sometimes. Hence, there is a need for a protective cover for operating room instruments to provide a protective barrier from fluids present in an operating room, which will decrease the likelihood that an instrument will be contaminated or damaged by exposure to such operating room fluids. Likewise, there is a need for a protective cover for operating room instruments that will also provide a sterile barrier to the surgical patient from possible organisms or contaminants that may gather on an instrument over time.

Sometimes the cameras and microscope lenses used in an operating room are attached to an elongated arm expending above or over the operating table to provide easy access to the instrument without obstructing the ground area around operating table. Also other types of instruments, such as power drills, power saws, suction lines, water hoses, x-ray components, ultrasonic transducers, or laser wands, may extend from elongated arms for the same reason. Furthermore, even when surgical or diagnostic instruments are not extending from an elongated arm above the operating table, such instruments often have long extension lines between a main unit positioned away from the operating table and the tool end or lens where the instrument is performing its task at the operating table. Hence, there is a need for a protective cover for surgical and diagnostic instruments having elongated support arms or elongated extensions.

Because the contaminates and fluids found in an operating room environment are often biohazardous, a protective cover for elongated surgical or diagnostic instruments needs to be disposable or removable for sanitizing. Hence, there is a need for a protective cover for elongated instruments that is easily installed or removed, as well as being inexpensive and disposable.

Often there is a need to quickly clean and disinfect an operating room in preparation for a subsequent surgery. Hence much of the instruments, furniture, and personnel in an operating room may be covered with readily removable covers. However, such covers often need to be securely affixed during surgery and need to be without large sagging portions that may obstruct or trip personnel in the operating room. Thus, there is a need for a protective cover for elongated instruments that is quick and easy to install and remove, while also being securely affixed and non-obstructive during use.

In addition to surgery rooms, there is another environment where cleanliness is crucial: computer component fabrication and manufacturing. Computer technology advancements are often attributed to advancements in fabrication processes that allow for ever smaller components to be made, and for more pure or precise material structures to minimize the number of defects at a molecular level. Fabrication facilities for producing computer components are sometimes even more sterile and clean than an operating room. Often the tools and instruments used in such fabrication facilities have elongated portions or extensions, such as robotic arms where elongated instruments are used in a clean or sterile environment. The computer component industry is just one example among many other fields. Hence, there are many other industries and fields where cleanliness or protection may be needed for an elongated instrument. Thus, there is also a need in other industries and fields for a protective cover for an elongated instrument.

### BRIEF SUMMARY OF THE INVENTION

The problems and needs outlined above are largely solved and met by the present invention. The following are some example objects, among many, of the present invention.

It is an object of the present invention to provide a protective cover for surgical and diagnostic instruments having elongated support arms, elongated extensions, and/or power and communications cords extending therefrom.

It is another object of the present invention to provide a protective cover for an elongated instrument that can provide a protective barrier from fluids present in an operating room, thereby decreasing the likelihood that the instrument will be contaminated or damaged by exposure to operating room fluids.

It is yet another object of the present invention to provide a protective cover for an elongated instrument that can provide a sterile barrier to a surgical patient from possible organisms or contaminants that may gather on an instrument in an operating room over time.

It is still another object of the present invention to provide a protective cover for elongated instruments that is easily installed, as well as being inexpensive and disposable.

It is another object of the present invention to provide a protective cover for an elongated instrument that is quick and easy to install and remove, while also being securely affixed and non-obstructive during use.

It is a further object of the present invention to provide a protective cover for an elongated instrument that is adaptable to various fields and industries.

In accordance with one aspect of the present invention, a protective cover for an elongated instrument is provided, which comprises a hollow elongated member and a loop member. The hollow elongated member has a first and second end at opposite ends thereof. At least a portion of the elongated member is a pliable material. The loop member extends along a generally circumferential direction of the elongated member at the first end. The loop member is affixed to and supports the elongated member along at least a portion of a circumference of the elongated member such that an opening is formed at the first end of the elongated member, and such that when in a relaxed configuration, the opening remains open without a force being applied to the loop member. Also, there can be a plurality of loop members.

In accordance with another aspect of the present invention, a protective cover for an elongated instrument is provided, which comprises a hollow elongated member and a loop member. The hollow elongated member has a first and second end at opposite ends thereof. At least a portion of the elongated member is a pliable material. The loop member extends along a perimeter of the elongated member at the first end. The loop member is affixed to and supports the elongated member along at least a portion of the perimeter of the elongated member such that an opening is formed at the first end of the elongated member. At least a portion of the loop member has a cross-section with a height dimension of the cross-section being at least ⅛ the size of a longitudinal dimension of the cross-section.

In accordance with yet another aspect of the present invention, a protective cover for an elongated instrument is provided, which comprises a pliable, generally tube-shaped portion and a pair of ring members. The tube-shaped portion has a first and second end. The pair of ring members are affixed to the tube-shaped portion at the first end. The ring members are separated by a spaced distance along a longitudinal extent of the tube-shaped portion such that a hand-hold region is formed at the first end. A first, generally round opening is formed at the first end by the ring members when the ring members are in a relaxed state. A tip of the second end is adapted to form a second opening when the elongated instrument is operably placed in the protective cover.

### BRIEF DESCRIPTION OF THE DRAWINGS

Other objects and advantages of the invention will become apparent upon reading the following detailed description and upon referencing the accompanying drawings, in which:

FIG. 1 shows a protective cover in an extended configuration in accordance with a first embodiment of the present invention;

FIG. 2A shows the protective cover of FIG. 1 in a folded configuration and a person inserting an elongated instrument into the protective cover;

FIG. 2B shows the protective cover of FIG. 1 being used to protect an elongated instrument in an operating room;

FIG. 3 shows the protective cover of FIG. 1 being used to protect another elongated instrument in an operating room;

FIG. 4 shows a protective cover in an extended configuration in accordance with a second embodiment of the present invention;

FIGS. 5a-5d each shows a second end portion of a protective cover in accordance with a third through sixth embodiment, respectively, of the present invention;

FIGS. 6a-6b each shows a first end portion of a protective cover in accordance with a seventh and eighth embodiment, respectively, of the present invention;

FIGS. 7a-7c each shows a protective cover in an extended configuration in accordance with a ninth through eleventh embodiment, respectively, of the present invention;

FIGS. 8a-8e each shows a first end portion of a protective cover in accordance with a twelfth through sixteenth embodiment, respectively, of the present invention;

FIGS. 9a-9i each is a cut-away, partial view showing a cross-section of a loop member for a protective cover in accordance with a seventeenth through twenty-fifth embodiment, respectively, of the present invention;

FIGS. 10a-10c each shows a loop member of a protective cover in accordance with a twenty-sixth through twenty-eighth embodiment, respectively, of the present invention;

FIG. 11 shows a loop member and a phantom line view of a first end of a protective cover in accordance with a twenty-ninth embodiment of the present invention;

FIG. 12 shows a loop member and a phantom line view of a first end of a protective cover in accordance with a thirtieth embodiment of the present invention;

FIG. 13 shows a loop member and a phantom line view of a first end of a protective cover in accordance with a thirty-first embodiment of the present invention; and

FIG. 14 shows a protective cover in an extended configuration in accordance with a thirty-second embodiment of the present invention.

### DETAILED DESCRIPTION OF THE INVENTION

Referring now to the drawings, wherein like reference numbers are used to designate like elements throughout the various views, several embodiments of the present invention are further described and illustrated. In some instances the drawings have been exaggerated and/or simplified in places for illustrative purposes only. One of ordinary skill in the art will appreciate the many possible applications and variations of the present invention based on the following examples of possible embodiments of the present invention.

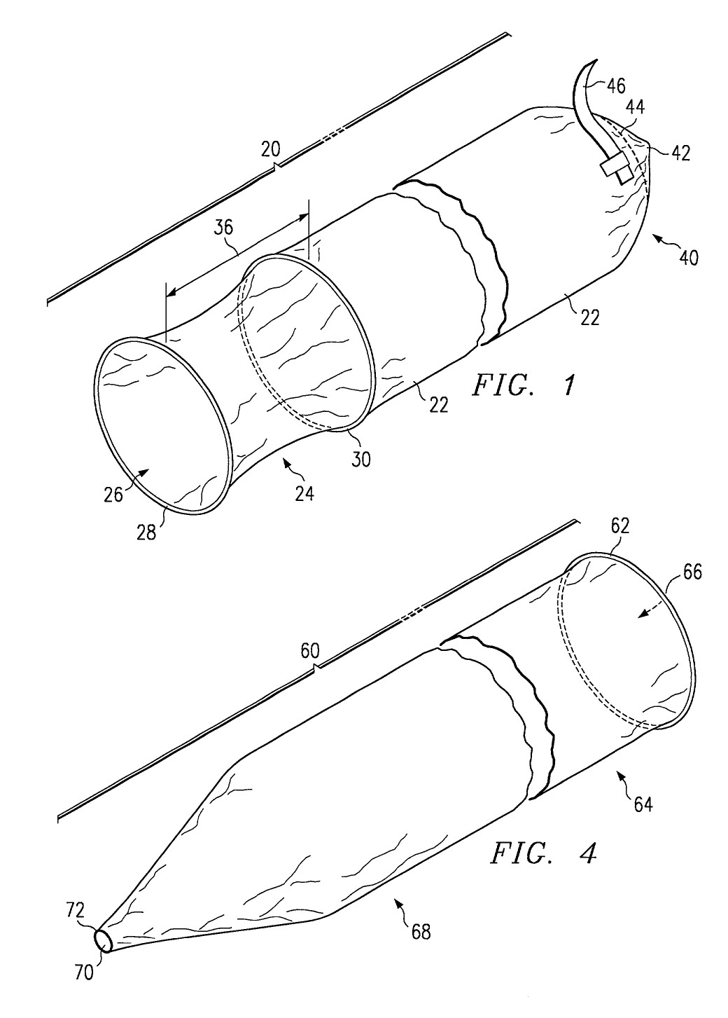

FIGS. 1-3 show a protective cover 20 in accordance with a first embodiment of the present invention. The protective cover 20 shown in FIG. 1 is in an extended configuration, but the middle section is not shown for illustration purposes. The protective cover 20 is about eight feet long along the longitudinal direction with a diameter of about five inches, but these sizes may vary for different applications. The protective cover has a hollow, elongated member 22 made from a pliable material. The elongated member 22 has a first end 24 with a first opening 26. The first opening 26 is maintained by a pair of loop members 28, 30 at the first end 24. The loop members 28, 30 extend along a generally circumferential direction of elongated member 22. Each loop member 28, 30 is affixed to and supports the elongated member 22 along the circumferential direction such that the first opening 26 remains open when the loop members are in a relaxed configuration. Hence, there is no need for an eternal force to be applied the loop members 28, 30 for maintaining the first opening 26.

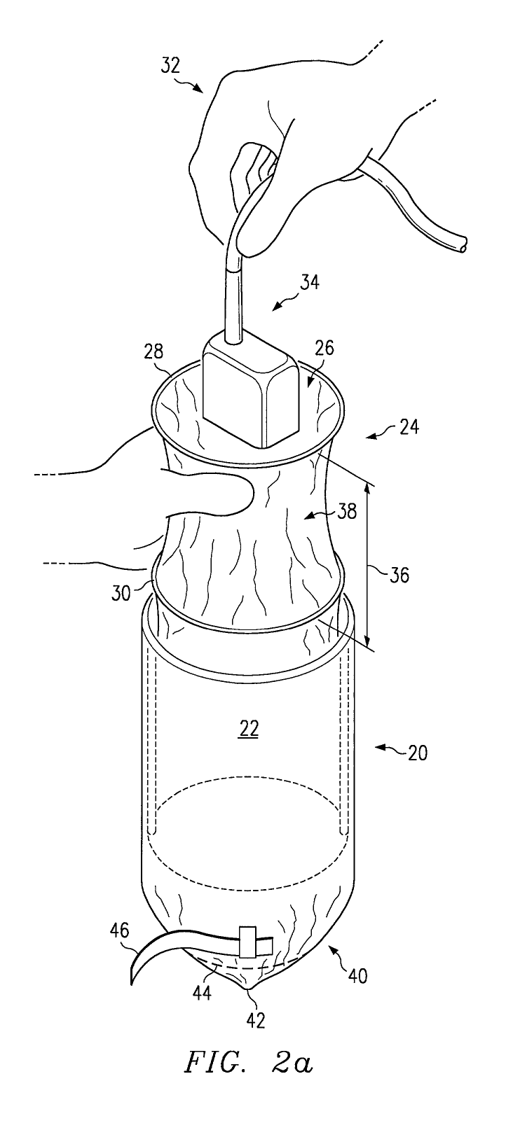

FIG. 2A shows a person 32 inserting an elongated instrument 34 into the first opening 26 of the protective cover 20 at the first end 24. As shown in FIG. 2A, the loop members 28, 30 are separated by a spaced distance 36 along a longitudinal extent of the elongated member 22 such that a hand-hold region 38 is formed between the loop members. The hand-hold region 38 makes it easier to handle the protective cover 20 when inserting an elongated instrument 34. The loop members 28, 30 can also provide a handle for pulling or draping the protective cover 20 over an elongated arm of an instrument or over a long extension of an instrument, while also maintaining the first opening 26. Such handles and hand-hold regions formed by the loop members 28, 30 are especially useful when a person is wearing gloves, which may restrict the movement or abilities of a person's hands. Also, such handles and hand-hold regions may allow a person to apply the protective cover 20 with one hand while holding or inserting an instrument with the other hand. Furthermore, such handles or hand-hold regions will likely increase the ease and speed at which a protective cover 20 can be installed or removed.

The elongated member 22 has a second end 40 that is tapered to a tip 42. The tip 42 of the second end 40 is perforated around a circumference of the second end so that the tip can be easily removed by tearing along the perforated line 44. Removal of the tip 42 creates a second opening at the second end 40, which allows an elongated instrument to extend outside of the protective cover 20 at the second end. A removable strip of adhesive tape 46 is affixed to the second end 40. The tape 46 can be used to secure or seal the second end 40 after an instrument is positioned within the second opening.

FIG. 2B shows the protective cover 20 according to the first embodiment being used to cover the elongated instrument 34 while the person 32 is using the instrument 34 in an operating room environment. Although the elongated instrument 34 has a power and communications cord 35 extending across the floor to a larger unit 47 that drives the handheld portion of the instrument 34, the protective cover provides protection for the elongated instrument 34 to help keep it clean.

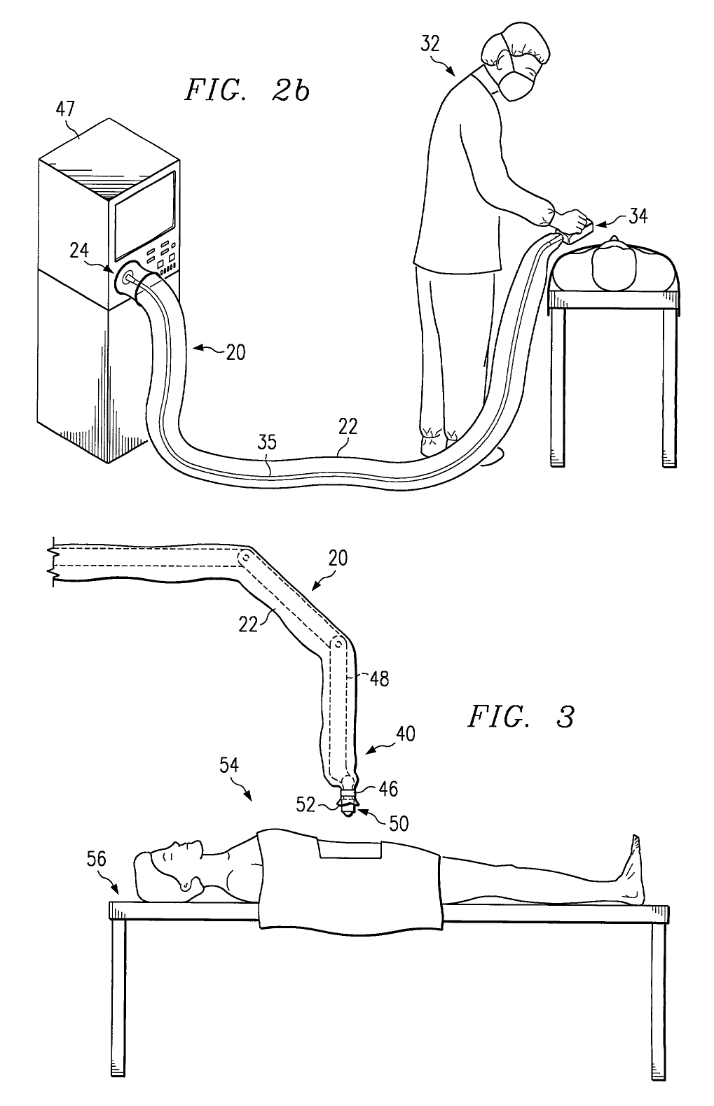

FIG. 3 shows the protective cover 20 according to the first embodiment being used to cover an elongated support arm 48 for an instrument 50 in an operating room (the first end 24 is not shown in FIG. 3). With the instrument 50 extending through the second opening 52 of the second end 40, the tape 46 is used to gather the second end closely around the end of the instrument, thereby securing the second end to the instrument during use of the instrument. Also, if secured tightly, the tape 46 can seal the second opening 52 to prevent contaminants from entering the second opening. Hence, the instrument 50 is protected from contaminants and the patient 54 being operated on is protected from debris, contaminants, or organisms that may be on the instrument. When the protective cover 20 is operably installed on an instrument, the first end 24 often will not need to be sealed or secured if the first end is far from the operating table 56 or if the first end is draped downward.

There can be many variations of the present invention, some of which will be illustrated by embodiments shown and described herein. For example, there need not be two loop members. FIG. 4 shows a protective cover 60 in accordance with a second embodiment of the present invention. The protective cover 60 has one loop member 62 at its first end 64 to provide and maintain a first opening 66 at the first end. Also, the second embodiment shown in FIG. 4 has a different second end 68 than the first embodiment. The second end 68 is tapered over a longer longitudinal distance. A second opening 70 is pre-formed at the second end tip 72 and there is no adhesive tape affixed to the second end 68. The second embodiment could be sized for a particular application so that the pre-formed second opening 66 is approximately the same diameter or smaller than that of an instrument tip. Hence, the second embodiment can be adapted so that the second opening 66 must be stretched open slightly to allow the instrument tip to extend through it. Thus, in such a case, the second end tip 72 would fit snuggly onto the instrument tip to seal and secure the second opening 66. In another application, the means of securing and/or sealing the second end 68 may be provided by any of other commonly known means, including but not limited to: a hose clamp, a twist tie, a zip tie, a rubber band, a zipper, a snap, a button, a belt, velcro, heat shrinking, or tape from a roll or dispenser.

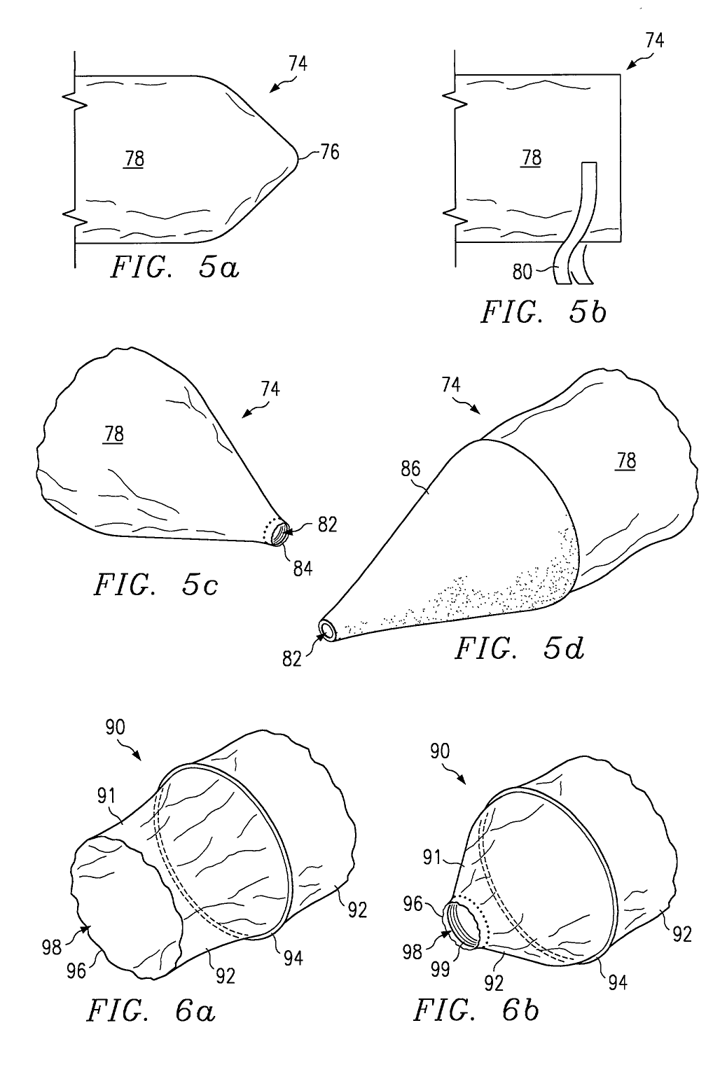

FIGS. 5a-5d show some other possible variations, among many, of a second end 74 for the present invention (the first end is not shown in FIGS. 5a-5d and may vary). In a third embodiment shown in FIG. 5a, the second end 74 is tapered to a tip 76, but without an opening or perforation. Hence, the second end 74 is closed or has a closed tip. There may be some applications where an instrument will work within the protective cover without extending the instrument through the second end 74. For example, if the elongated member 78 is made from a translucent or transparent material, such as clear plastic, then a camera lens can see through the protective cover and may not need to extend out of the second end 74, depending on the resolution and image quality required. Also, the tip 76 of the second end 74 may be adapted to allow an instrument tip to be forced through the second end 74 to form a second opening by making the elongated member 78 from pliable sheet plastic, for example, or by making the material thinner at the tip 76 of the second end.

In a fourth embodiment shown in FIG. 5b, the second end 74 is blunt without a pre-formed second opening. Hence, the fourth embodiment has a closed second end 74. The second end 74 has tape 80 partially affixed thereto. Thus, as with the embodiment shown in FIG. 5a, the second end 74 of FIG. 5b may be adapted to allow an instrument to operate within the protective cover without extending from the second end. Also, the second end 74 in FIG. 5b may be adapted for forcing an instrument tip through the second end at any location of the second end, followed by gathering, securing, and/or sealing a second opening formed in the second end with the tape 80 attached to the second end.

In a fifth embodiment shown in FIG. 5c, the second end 74 has an expandable second opening 82. The second opening 82 is lined with an elastic member 84, which gathers the second end 74 and makes the second opening smaller when the elastic member is in a contracted configuration. When an instrument is extended through the second opening 82, the second opening can be expanded by expanding the elastic member 84. Hence, depending on the application, the elastic member 84 may secure the second end 74 and seal the second opening 82 such that no other securing or sealing means are needed at the second end.

In a sixth embodiment shown in FIG. 5d, the second end 74 has a thermal elastic polymer section 86, such as polyurethane, that forms a second opening 82 at the tip. The thermal elastic polymer section 86 allows for elasticated fenestration. The second opening 82 can be sized for a given instrument such that the instrument fits snuggly within it and extends partially outside of the protective cover. Thus in an embodiment of the present invention, the elongated member 78 may comprise sections or portions having different materials along any portion of the protective cover, which may vary to suit a given application.

In the present invention, even though the loop member is at the first end, it need not be at the most distal end of the first end. FIGS. 6a and 6b show some other possible variations, among many, of a first end 90 for the present invention (the second end is not shown in FIGS. 6a and 6b, and may vary). In a seventh embodiment shown in FIG. 6a, a portion 91 of the elongated member 92 extends beyond the loop member 94 toward the most distal end 96 of the first end 90. Such distal extending portion 91 may be used to gather, secure, and/or seal the first opening 98 after the protective cover is placed on an instrument. For example, the distal extending portion 91 can be gathered and taped tightly to the instrument to secure and seal the first opening 98 (this configuration not shown). As shown in an eighth embodiment in FIG. 6b, the first opening 98 at the distal extending portion 91 of the first end 90 can have an elastic member 99 affixed to the distal extending portion 91 inside the first opening to allow the first opening to be smaller yet still expandable for inserting an instrument.

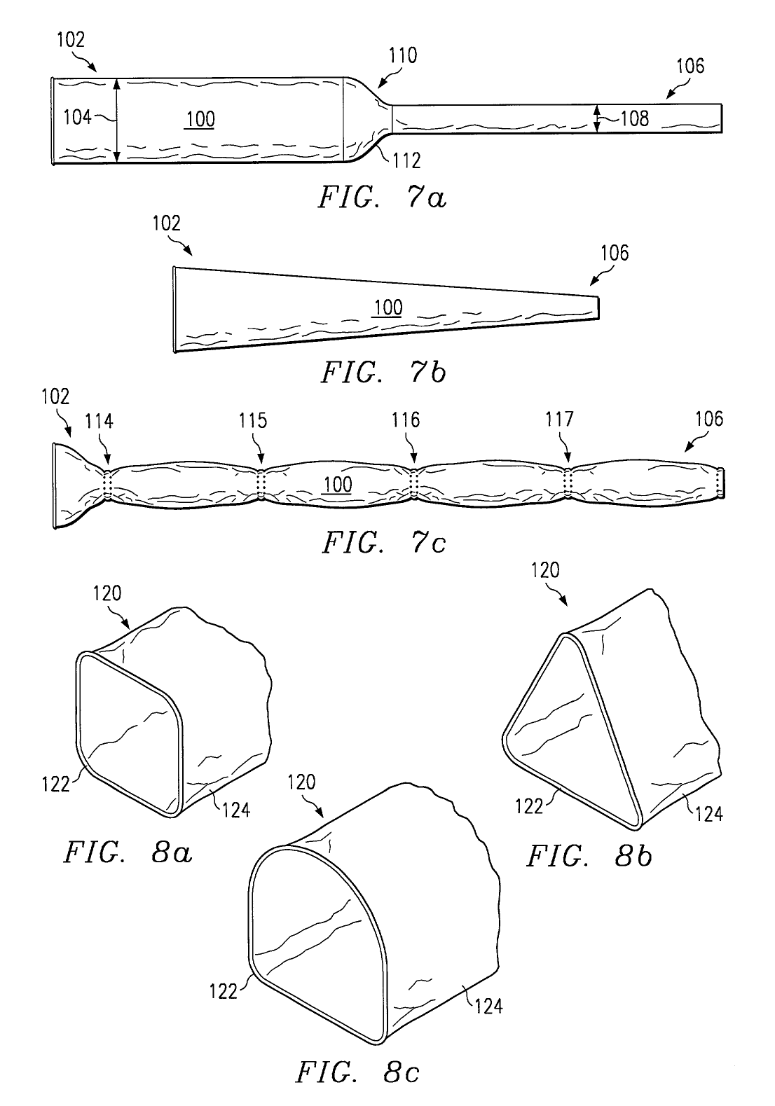

In the present invention, an elongated member 100 can have a variety of shapes and diameters. In a ninth embodiment shown in FIG. 7a, the first end 102 has a first diameter 104, the second end 106 has a second diameter 108, and a middle portion 110 between the first and second ends 102, 106 has a tapered transition 112 from the first diameter 104 to the second diameter 108. In a tenth embodiment shown in FIG. 7b, the first end 102 has a larger diameter than the second end 106, and the elongated member 100 is generally conical-shaped. In an eleventh embodiment shown in FIG. 7c, the elongated member 100 has a series of elastic regions 114, 115, 116, 117 along its longitudinal extent. Hence, the elongated member 100 is expandable at each elastic region 114, 115, 116, 117. Incorporating the elastic regions 114, 115, 116, 117 can prevent the protective cover from sagging or hanging below a horizontal extension of an instrument, which helps ensure that the elongated member 100 does not become obstructive while in use. Also, the diameter of the elongated member 100 can be constant along all or most of its longitudinal extent.

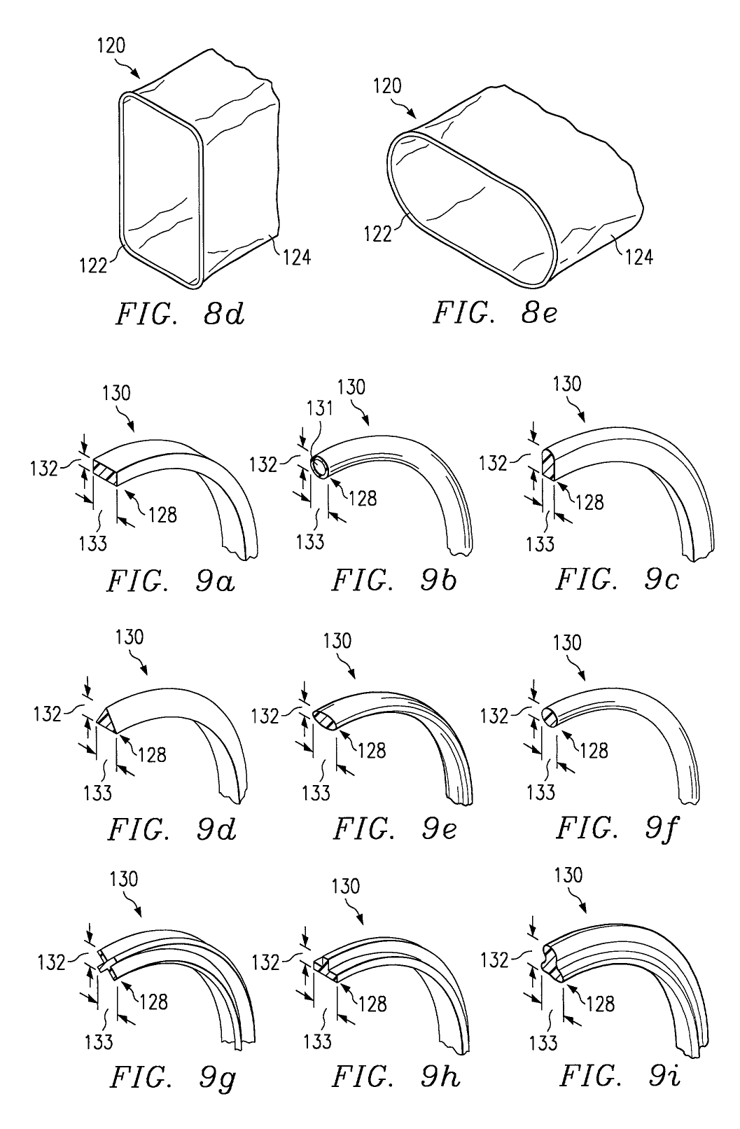

In the present invention, there can be many variations in the shape of a loop member at the first end 120, some of which are illustrated by the embodiments shown and described in FIGS. 8a-8e (the second end is not shown in FIGS. 8a-8e and may vary). As shown in a twelfth embodiment in FIG. 8a, a loop member 122 can be generally square or diamond shaped. Also, a loop member 122 can be generally triangular shaped, generally D-shaped, or generally rectangular shaped, as shown in FIGS. 8b, 8c, and 8d, respectively. FIG. 8e shows a sixteenth embodiment having a generally elliptical or oval shaped loop member 122. As also illustrated in the embodiments shown in FIGS. 8a-8e, the shape of a loop member 122 may determine or influence the general cross-sectional shape of the elongated member 124 along its longitudinal extent. It should be noted also that in an embodiment having more than one loop member (e.g., two loop members, three loop members, etc.), the loop members need not have the same shape nor the same opening size.

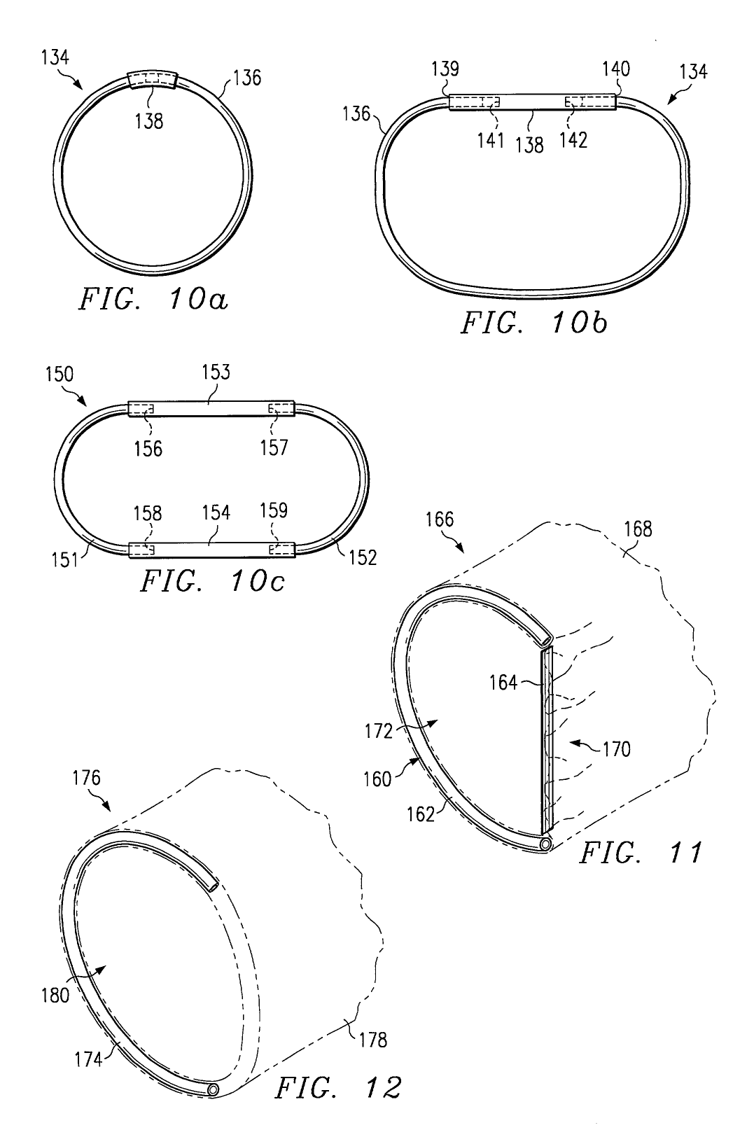

In the present invention, there can be many shape variations for a cross-section 128 of a loop member 130, some of which are illustrated by the embodiments shown and described in FIGS. 9a-9i (only a portion of a loop member 130 is shown in each of FIGS. 9a-9i for illustrating the cross-section variations). Many of the possible cross-section shape variations can have polygonal shapes, both regular and non-regular polygons. In a seventeenth embodiment shown in FIG. 9a, the cross-section 128 of a loop member 130 has a solid rectangular shape. In an eighteenth embodiment shown in FIG. 9b, the cross-section 128 of the loop member 130 has a round annular shape with a hollow center region 131. Hence, the loop member 130 in FIG. 9b is tubular. In other possible embodiments, the cross-section 128 of a loop member 130 can have a D-shape, a triangular shape, a generally elliptical shape, a circular shape, an X-shape, a T-shape, or an arbitrary non-regular shape, as shown in FIGS. 9c-9i respectively.

As shown in FIGS. 9a-9i, a cross-section 128 of at least a portion of a loop member 130 in some embodiments of the present invention has a height dimension 132 of the cross-section 128 being at least ⅛ the size of a longitudinal dimension 133 of the cross-section 128. For example, if the longitudinal dimension 133 of a cross-section 128 is 0.8 inch, then the height dimension 132 will be at least 0.1 inch (0.8 inch×⅛=0.1 inch). Such proportions provided by this size relationship between the height dimension 132 and the longitudinal dimension 133 helps to provide the strength in the loop member 130 needed to keep the first opening 26 at the first end 24 open without applying additional force to the loop member 130. In other words, it helps to provide the structural stability needed to form the first opening 26 with the loop member 130, even in a relaxed state.

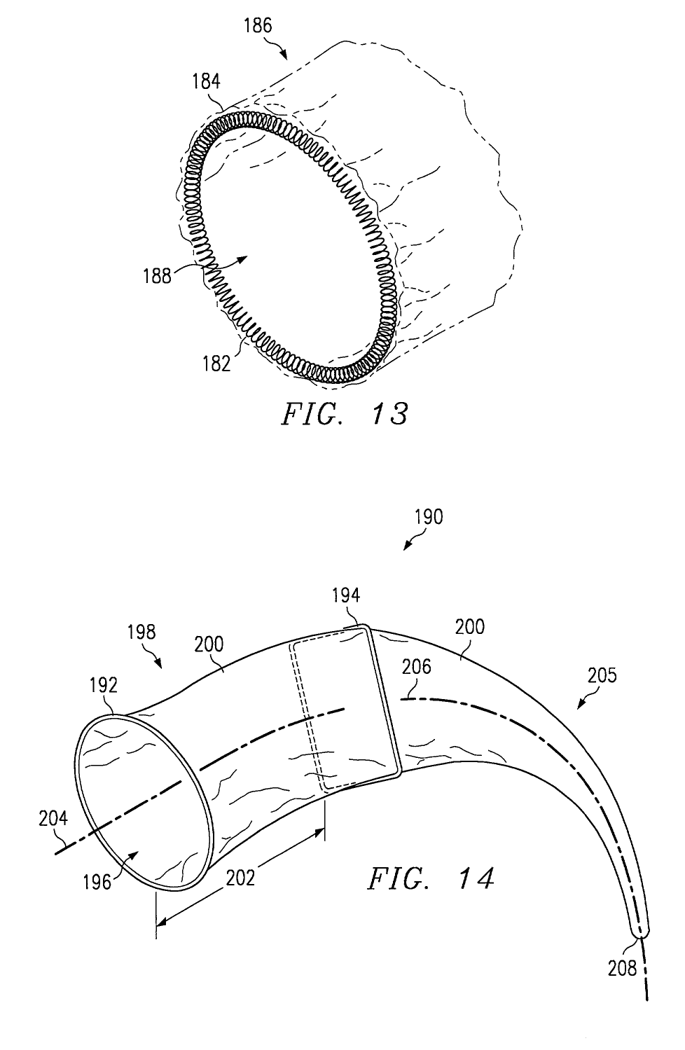

In the present invention, a loop member can be one continuous piece or it can comprise a plurality of pieces. Also, a loop member can be rigid or flexible, as long as the loop member provides enough support to maintain a first opening at a first end of a protective cover. Furthermore, in a multi-piece loop member there can be flexible and rigid pieces combined. Some examples of possible multi-piece loop members are shown in FIGS. 10a-11 (only loop members are shown in FIGS. 10a-10c).

In a twenty-sixth embodiment shown in FIG. 10a, a loop member 134 has a first piece 136 forming a substantial portion of the loop member. The first piece 136 is a hollow, flexible plastic tube. A second piece 138 of the loop member 134 is a tube section having a larger diameter than the first piece 136, such that the distal ends of the first piece can be looped around to form a circle and inserted into the distal ends of the second piece. The resulting structure when the first and second pieces 136, 138 are combined is a generally circular shaped loop member 134, as shown in FIG. 10a.

In a twenty-seventh embodiment shown in FIG. 10b, a loop member 134 is formed with two pieces 136, 138 similar to the embodiment shown in FIG. 10a. The first piece 136 is a solid, flexible plastic bar. The second piece 138 is also a solid, plastic bar, but the second piece 138 is more rigid than the first piece 136. Also, the second piece 138 has a larger diameter than the first piece 136. Each distal end 139, 140 of the second piece 138 has a flat-bottomed bore 141, 142 formed therein, such that the distal ends of the first piece 136 can be looped around and inserted snuggly into the bores 141, 142 of the second piece 138. The resulting structure when the first and second pieces 136, 138 are combined is a generally D-shaped loop member 134, as shown in FIG. 10b.

In a twenty-eighth embodiment shown in FIG. 10c, a loop member 150 is formed with four pieces 151, 152, 153, 154. The four pieces comprise a pair of hollow, flexible plastic tubes 151, 152 of about the same length, and a pair of solid plastic bars 153, 154 of about the same length. The pair of bars 153, 154 are more rigid and have a larger diameter than the pair of tubes 151, 152. Each bar of the pair of bars 153, 154 has a bore 156, 157, 158, 159 formed in each of its distal ends, such that the distal ends of the pair of tubes 151, 152 can be snuggly inserted into the bores 156, 157, 158, 159 of the pair of bars 153, 154. The resulting structure when the four pieces 151, 152, 153, 154 are combined is a generally oval shaped loop member 150, as shown in FIG. 10c.

In twenty-ninth embodiment shown in FIG. 11, a loop member 160 is formed by a C-shaped rigid tube 162 and an elastic member 164. A portion of a first end 166 of an elongated member 168 is shown in phantom lines for illustrative purposes. The elastic member 164 extends within the open side 170 of the C-shaped structure of the tube 162. When affixed to the elongated member 168, the C-shaped tube 162 and the elastic member 164 are held in their respective configurations shown in FIG. 11. The C-shaped tube 162 supports and maintains a first opening 172 in the first end 166 of the elongated member 168, and the elastic member 164 allows the first opening to be expandable. Hence, a protective cover in accordance with the twenty-eighth embodiment can accommodate a variety of instrument sizes because it is expandable.

In the present invention, the loop member need not extend around an entire circumference of an elongated member to maintain and support a first opening. For example, FIG. 12 shows a thirtieth embodiment having a flexible, C-shaped bar 174 as a loop member. A first end 176 of an elongated member 178 is shown in phantom lines for illustrative purposes. The C-shaped bar 174 need only extend around the circumference of the elongated member 178 enough to provide support and maintain a first opening 180, which will depend on the flexibility of the loop member and pliability of the elongated member at the first opening.

FIG. 13 shows yet another possible variation of a loop member in a thirty-first embodiment of the present invention, which is a coiled-spring member 182. A first end 184 of an elongated member 186 is shown in phantom lines for illustration purposes. The coiled-spring structure of the loop member 182 allows a first opening 188 formed at the first end 184 to be expandable. Hence, a protective cover in accordance with the thirtieth embodiment can also accommodate a variety of instrument sizes because it is expandable.

When there are a plurality of loop members, the loop members need not be parallel to one another. Also, a loop member need not extend along a circumference of an elongated member on a plane perpendicular to a longitudinal axis of the elongated member. In accordance with a thirty-second embodiment of the present invention, FIG. 14 shows a protective cover 190 for an elongated instrument having two loop members 192, 194. The first loop member 192 is circular shaped forming a first opening 196 at a first end 198 of an elongated member 200. The second loop member 194 at the first end 198 is rectangular shaped and separated from the first loop member 192 by a space distance 202. The second loop member 194 extends along a generally circumferential direction that is not on a plane perpendicular to a longitudinal axis 204 of the elongated member 200, and the second loop member 194 is not parallel to the first loop member 192. Also, as shown in FIG. 14, an elongated member need not be formed in a straight line. The second end 205 of the elongated member 200 extends along a generally curved axis 206. Hence, to better fit a given elongated instrument, the longitudinal shape or configuration of an elongated member may vary for a given application. In FIG. 14, the tip 208 of the second end 205 is rounded and without a pre-formed opening.

For packaging and ease of handling, a protective cover in accordance with the present invention may be folded or rolled in a variety of ways, as is conventionally known for similarly shaped structures. FIG. 2 shows the protective cover 20 in accordance with the present invention that is telescopically folded. Hence, as an instrument is inserted into a protective cover or as the protective cover is draped over an instrument, the protective cover can be easily unfolded, which can prevent the protective cover from being contaminated by touching the floor. Also, folding or rolling a protective cover provides a way of packaging the protective cover in a more compact or flat package configuration.

In the present invention, a variety of materials (sterile or non-sterile) for an elongated member can be used, including but not limited to: non-woven material, synthetic latex-like polymers, transparent sheet plastic, latex, rubber, plastic, clothe, or any combination thereof. A portion of an elongated member must be pliable to allow the elongated member to bend or flex as needed for a given application. Thus, a portion of an elongated member may not be pliable. However, in most applications, a majority or all of an elongated member will likely be pliable sheet plastic to enable a protective cover to be versatile, lightweight, foldable or rollable, disposable, and inexpensive. Likewise, a variety of materials can be used for a loop member, including but not limited to: plastic, wood, metal, rubber, or any combination thereof. It should also be noted that further embodiments of the present invention can be formed by any combination of the previously discussed embodiments and variations.

It will be appreciated by those skilled in the art having the benefit of this disclosure that the present invention provides a protective cover for an elongated instrument. It should be understood that the drawings and detailed description herein are to be regarded in an illustrative rather than a restrictive manner, and are not intended to limit the invention to the particular forms and examples disclosed. On the contrary, the invention includes any further modifications, changes, rearrangements, substitutions, alternatives, design choices, and embodiments apparent to those of ordinary skill in the art, without departing from the spirit and scope of this invention, as defined by the following claims. Thus, it is intended that the following claims be interpreted to embrace all such further modifications, changes, rearrangements, substitutions, alternatives, design choices, and embodiments.

What is claimed is:

1. A protective cover for an elongated instrument comprising:
a hollow elongated member having a first and second end at opposite ends thereof, at least a portion of said elongated member being a pliable material; and
a loop member extending along a generally circumferential direction of said elongated member at said first end, said loop member being affixed to and supporting said elongated member along at least a portion of a circumference of said elongated member such that an opening is formed at said first end of said elongated member, and such that when in a relaxed configuration, said opening remains open without a force being applied to said loop member.

2. A protective cover in accordance with claim 1, further comprising:
a second loop member extending along said generally circumferential direction of said elongated member, said second loop member being affixed to and supporting said elongated member along at least a portion of another circumference of said elongated member.

3. A protective cover in accordance with claim 2, wherein said loop members are separated by a spaced distance along a longitudinal extent of said elongated member such that a hand-hold portion is formed between said loop members.

4. A protective cover in accordance with claim 1, wherein said second end of said elongated member is tapered.

5. A protective cover in accordance with claim 4, wherein said second end of said elongated member has a removable perforated tip.

6. A protective cover in accordance with claim 4, wherein said second end of said elongated member has a closed tip.

7. A protective cover in accordance with claim 4, wherein said second end of said elongated member has an expandable second opening formed at the tip of said second end.

8. A protective cover in accordance with claim 1, wherein said elongated member is telescopically folded for packaging and handling.

9. A protective cover in accordance with claim 1, wherein said loop member is flexible.

10. A protective cover in accordance with claim 1, wherein said loop member is rigid.

11. A protective cover in accordance with claim 1, wherein a cross-section of said loop member has a polygonal shape.

12. A protective cover in accordance with claim 11, wherein said polygonal shape is selected from one of a triangular, square, rectangular, pentagonal, hexagonal, octagonal, trapezoidal, and non-regular shape.

13. A protective cover in accordance with claim 1, further comprising an adhesive member removably affixed at said second end of said elongated member.

14. A protective cover in accordance with claim 1, wherein said loop member comprises multiple segments.

15. A protective cover in accordance with claim 1, further comprising:
an elastic member affixed to said elongated member at said first end, such that said opening at said first end is expandable.

16. A protective cover for an elongated instrument comprising:
a hollow elongated member having a first and second end at opposite ends thereof, at least a portion of said elongated member being a pliable material; and
a loop member extending along a perimeter of said elongated member at said first end, said loop member being affixed to and supporting said elongated member along at least a portion of said perimeter of said elongated member such that an opening is formed at said first end of said elongated member, wherein at least a portion of said loop member has a cross-section with a height dimension of said cross-section being at least ⅛ the size of a longitudinal dimension of said cross-section.

17. A protective cover in accordance with claim 16, further comprising:
a second loop member extending along another perimeter of said elongated member, said second loop member being affixed to and supporting said elongated member along at least a portion of said another perimeter of said elongated member.

18. A protective cover in accordance with claim 17, wherein each of said loop members has a round cross-section, has a ring configuration, is separated from each other by a spaced distance along a longitudinal extent of said elongated member to provide a hand-hold area therebetween, and is made of a flexible material.

19. A protective cover in accordance with claim 17, wherein said pliable material of said elongated member is sheet-like, material.

20. A protective cover in accordance with claim 16, wherein said second end of said elongated member is tapered.

21. A protective cover in accordance with claim 20, wherein said second end of said elongated member has a removable perforated tip.

22. A protective cover in accordance with claim 20, wherein said second end of said elongated member has a closed tip.

23. A protective cover in accordance with claim 20, wherein said second end of said elongated member has an expandable second opening formed at the tip of said second end.

24. A protective cover in accordance with claim 16, wherein said elongated member is telescopically folded for packaging and handling.

25. A protective cover in accordance with claim 16, wherein said cross-section of said loop member has a polygonal shape.

26. A protective cover in accordance with claim 25, wherein said polygonal shape is selected from one of a triangular, square, rectangular, pentagonal, hexagonal, octagonal, trapezoidal, and non-regular shape.

27. A protective cover in accordance with claim 16, further comprising an adhesive member removably affixed at said second end of said elongated member.

28. A protective cover for an elongated instrument, comprising:
a pliable, generally tube-shaped portion having a first and second end;
a pair of ring members affixed to said tube-shaped portion at said first end, said ring members being separated by a spaced distance along a longitudinal extent of said tube-shaped portion such that a hand-hold region is formed at said first end;
a first, generally round opening formed at said first end by said ring members when said ring members are in a relaxed state; and
a tip of said second end being adapted to form a second opening when said elongated instrument is operably placed in said protective cover.
